(19) United States
(12) Patent Application Publication
(10) Pub. No.: US 2003/0229368 A1
(43) Pub. Date: Dec. 11, 2003

(54) Endoscopic surgical clip

(76) Inventor: Frank J. Viola, Sandy Hook, CT (US)

Correspondence Address: Paul R. Audet, U.S. Surgical, a Div. of Tyco Healthcare Group LP, 150 Glover Avenue, Norwalk, CT, 06856, US

(21) Appl. No.: 10/421,171

(22) Filed: Apr. 22, 2003

Related U.S. Application Data
(60) Provisional application No. 60/374,624, filed on Apr. 22, 2002.
(60) Provisional application No. 60/374,673, filed on Apr. 22, 2002.

(30) Foreign Application Priority Data
Apr. 22, 2003 (WO) PCT/US03/12643

(51) Int. Cl. A61B 17/08

(52) U.S. Cl. 606/158

(57) ABSTRACT

An endoscopic surgical clip for occluding vessels has a jaw comprised of a pair of arms that are connected to each other and are biased together by a spring section. The arms are elongate linear cantilevered beams extending beyond the end of the spring section. Compressing a portion of the spring section opens the arms. When the pressure is released, the bias of the spring section returns the arms to the first position.

### CROSS REFERENCE TO RELATED APPLICATIONS

This application claims priority from and the benefits of U.S. Provisional Application Serial Nos. 60/374,624 and 60/374,673 each filed on Apr. 22, 2002, the entire contents of each of which are incorporated herein by reference.

### BACKGROUND

1. Technical Field

The present disclosure relates to devices and methods for occluding vessels. More particularly, the present disclosure relates to endoscopic surgical clips and methods for using endoscopic surgical clips during surgical procedures.

2. Background of Related Art

During surgical procedures, the temporary or permanent occlusion of vessels is necessary to prevent the leakage of blood through incisions made at the surgical site. A wide variety of surgical ligating device configurations and techniques exist for accomplishing temporary and permanent occlusions. These include, for example, tubular, rod, and wire devices typically biased to a closed position. Ligating clips are configured for application directly by the hand of a surgeon, by remotely operated devices in open surgery, and/or by specialized instruments for minimally invasive surgical procedures.

Ligating clips used in minimally invasive surgery are frequently constrained in their configuration by their requirement to be fed in series into an instrument configured for remotely applying clips. The requirement for compatibility with the applying instrument often constrains the configuration of the clip. An example of such a clip is disclosed in U.S. Pat. No. 5,342,373 to Stefanchik et al. Stefanchik et al. relates to a sterile clip for ligating a vessel and a device for placing the sterile clip on the vessel.

Conventional ligating clips are generally formed from a ductile material that can adequately close on a vessel and remain in the as applied closed disposition. However, conventional clips, once applied, may at times be unable to respond to changes in thickness of the vessel wall due to swelling or inflammation, or to shrinkage when swelling or inflammation subsides. Thus, conventional ligating clips may traumatize the vessel or may allow some flow.

A need exists for a simplified ligating clip that can apply a range of compressive forces, can be readily applied directly by a surgeon, or can be applied remotely by a hand-held instrument during minimally invasive surgical procedures. A need further exists for a ligating clip that can respond to changes in vessel thickness after the ligating clip is applied to the vessel.

It is an object of the present disclosure to provide a surgical ligating clip that can apply a range of compressive forces to occlude a vessel.

It is another object of the present disclosure to provide a surgical ligating clip that can respond to changes in vessel thickness after it is applied.

### SUMMARY

This invention is directed to a surgical clip for occluding a vessel and that includes a jaw having first and second arms defining a longitudinal axis. Each arm includes a proximal end and a distal end, and an inner face and an outer face. A spring section biases the arms into a normal first position and communicates with the proximal ends of the jaw section. The inner face of the first arm abuts at least a portion of the inner face of the second arm and the spring section including at least one arcuate wall that defines at least arcuate loop. The inner face of the first arm can abut the inner face of the second arm substantially along the longitudinal axis. Each arm may include a socket disposed at the distal end of each arm. The inner faces of the first and second arms can be knurled, can have a sinusoidal pattern, or one where the sinusoidal pattern of the first arm is complementary to the sinusoidal pattern of the second arm. The spring section can be formed from a unitary continuous wall that forms the at least one arcuate loop and the at least one arcuate loop can be a bulbous loop having a closed loop end portion facing away from the jaw with an opposed end formed of converging first and second wall portions. One wall portion can communicate with the proximal end of the first arm and the other wall portion can communicate with the proximal end of the second arm. The inner face of the first arm can include a substantially convex portion directed toward the second arm, and the inner face of the second arm can include a juxtaposed substantially convex portion directed toward the first arm, where each of the convex portions are engaged with one another along the longitudinal axis of the arms. Alternately, the inner face of the first arm can include a substantially convex portion and the inner face of the second arm can include a complimentary substantially convex portion, where each of the convex portions can be engaged with one another along the longitudinal axis of the arms.

This invention is further directed to a surgical clip for occluding a vessel that includes a jaw having first and second elongated cantilevered beams that define a longitudinal axis, where each beam has a proximal end and a distal end. A spring section communicates with the proximal end of the beams for biasing the beams into a normal first position wherein the inner face of the first beam abuts at least a portion of the inner face of the second beam. The spring section includes first and second exterior arcuate members. The first exterior arcuate member is in communication with the proximal portion of the first beam and the second exterior arcuate member is in communication with the proximal portion of the second beam and the first and second exterior arcuate members form a third arcuate member therebetween. The first and second beams can includes an inner face and an outer face, where the inner face of the first beam can abut the inner face of the second beam substantially along the longitudinal axis. The first and second exterior arcuate members may include respective first and second loops, where the first and second loops each may have an opening facing the proximal ends of the first and second beams. The first and second exterior loops may form a third arcuate member in the form of a third loop, where the third loop can be located interior of the first and second loops and may have an opening that faces substantially 180° away from the first and second openings. The first, second, and third loops can be spaced apart from one another and can be transversely aligned. The first, second, and third loops may be flattened and may have substantially parallel sidewalls. The first, second, and third loops, and the jaw can be a continuous band of material.

This invention is also directed to a surgical clip for occluding a vessel including a jaw having first and second arms that define a longitudinal axis. Each arm has a proximal end and a distal end, and an inner face and an outer face. A spring section communicates with the proximal ends of the arms for biasing the arms into a normal first position wherein the inner face of the first arm abuts at least a portion of the inner face of the second arm. The spring section is U-shaped and formed of a unitary continuous wall that forms multiple contiguous bends, including first and second exterior U-shaped loops each having a convex exterior surface facing away from the jaw, and a third interior loop disposed between and extending from the first and second loops. The third interior loop includes an exterior concave surface facing the jaws, and the exterior walls of the first and second exterior loops extend into the first and second proximal end portions of the arms of the jaw. The first, second, and third loops can be formed by substantially U-shaped walls and may be spaced apart from each other. The portions of the substantially U-shaped walls can be substantially parallel to each other. The substantially U-shaped walls of the first, second, and third loops may be transversely aligned. The substantially parallel portions of the first and second exterior loops can communicate with a transitional wall portion, which can have an inward bend that extends toward and can engage the third interior loop. The first and second loops can be configured such that when the proximal end portions of the arms are opened against their bias, the first and second loops may move toward each other.

The presently disclosed endoscopic surgical clip, together with attendant advantages, will be best understood by reference to the following detailed description in conjunction with the figures below.

### BRIEF DESCRIPTION OF THE DRAWINGS

Preferred embodiments of the presently disclosed endoscopic surgical clip are described herein with reference to the drawings, wherein:

FIG. 1 is a perspective view of an embodiment of an endoscopic surgical clip in a first position in accordance with the present disclosure;

FIG. 1A is a perspective view of another embodiment of the endoscopic surgical clip in a first position in accordance with the present disclosure;

FIG. 1B is a perspective view of an alternative embodiment of the endoscopic surgical clip in a first position in accordance with the present disclosure;

FIG. 2 is a distal end view of the endoscopic surgical clip of FIG. 1 in a second position;

FIG. 3 is a distal end view of the endoscopic surgical clip of FIG. 1 in the first position with the endoscopic surgical clip occluding a vessel;

FIG. 4 is a perspective view of another embodiment of an endoscopic surgical clip in a second position in accordance with the present disclosure;

FIG. 5 is an end view of the endoscopic surgical clip of FIG. 4 in the first position with the endoscopic surgical clip positioned about a vessel;

FIG. 6 is an end perspective view with portions broken away of a first embodiment of the arms of the endoscopic surgical clip in accordance with the present disclosure;

FIG. 7 is an end perspective view with portions broken away of a second embodiment of the arms of the endoscopic surgical clip in accordance with the present disclosure;

FIG. 8 is an end perspective view with portions broken away of a third embodiment of the arms of the endoscopic surgical clip showing the tips of the arms in accordance with the present disclosure;

FIG. 9 is an end perspective view with portions broken away of a fourth embodiment of the arms of the endoscopic surgical clip showing the tips of the arms in accordance with the present disclosure; and

FIG. 10 is a perspective view with portions broken away of a fifth embodiment of the arms of the endoscopic surgical clip in the second position showing a knurled gripping surface in accordance with the present disclosure.

### DETAILED DESCRIPTION OF PREFERRED EMBODIMENTS

Preferred embodiments of the presently disclosed endoscopic surgical clip will now be described in detail with reference to the drawings, in which like reference numerals and characters designate identical or corresponding elements in each of the several views. As used herein, the term “distal” refers to that portion of the clip, or component thereof which is further from the user while the term “proximal” refers to that portion of the clip or component thereof which is closer to the user.

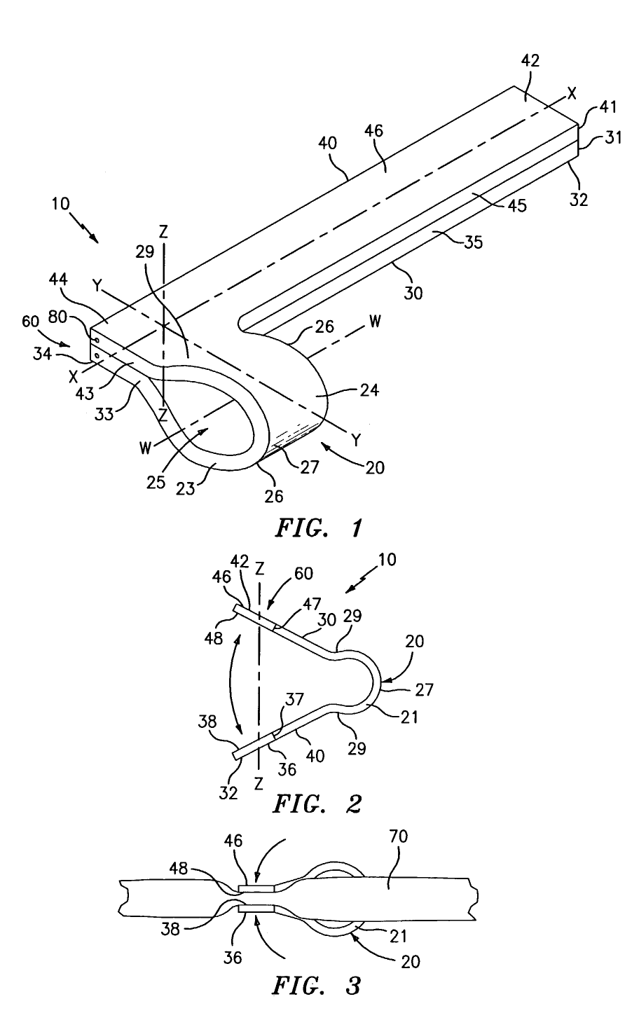

Referring now in specific detail to the drawings, and initially to FIGS. 1-3, an endoscopic surgical, or ligating clip 10 is shown constructed in accordance with an embodiment of the present disclosure. Ligating clip 10 includes a spring section 20 and a jaw 60. Jaw 60 includes a pair of beams, here shown as a first arm 30 and a second arm 40 each having a central longitudinal axis-X. An axis-Y is positioned through the center of spring section 20 and is substantially perpendicular to longitudinal axis-X. An axis-Z is positioned substantially perpendicular to and intersecting with, axes X and Y.

Spring section 20 has a nonlinear modified tubular-shaped loop 24 defining hole 25 substantially concentric with an axis-W and parallel with longitudinal axis-X. Bulbous loop 24 has a tubular wall 26 including an opposing pair of converging tapered arcuate portions 29 that transition or communicate into the proximal portions of arms 30 and 40. Tapered arcuate portions 29 have concave inner surfaces facing radially inward toward axis-W and exterior surfaces facing approximately radially outward from axis-W, the approximate center of loop 24. Axis-Y preferably is perpendicular to the longitudinal axes X and W. Spring section 20 is configured to provide a bias for urging arms 30 and 40 into direct contact defining a first, or closed, position. Loop 24 includes a first edge 21 (not shown in FIG. 1) and opposed second edge 23. When spring section 20 is viewed in a cross-section perpendicular to the longitudinal axis-X (See FIGS. 1 and 2), spring section 20 has a generally teardrop shape with a rounded bend of the teardrop defining a bulbous arcuate portion 27.

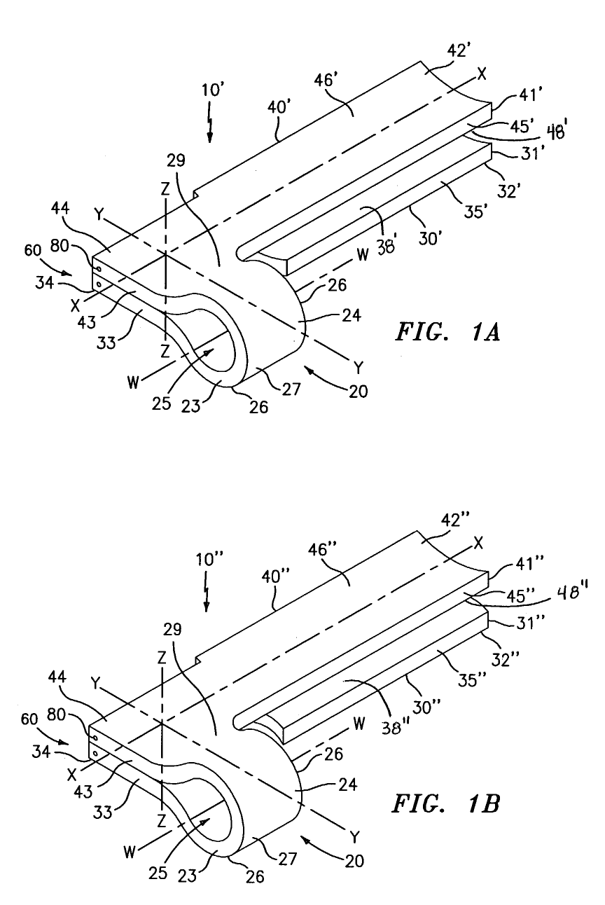

Arms 30 and 40 preferably are elongated and plate-like, extending substantially parallel with longitudinal axis-X and perpendicular to axis-Y. Although shown in FIGS. 1-3 as having flat surfaces, the surfaces and shapes of arms 30, 40 can any suitable shape and configuration. Arms 30 and 40 each have cantilevered distal end portions or tips 32 and 42, and proximal second end portions 34 and 44, connected to spring section 20. First end portions 32 and 42 preferably have straight angled ends having first edges 31 and 41, respectively, and generally parallel with axis-Y. However, first end portions 32, 42 can have other suitably shaped ends, e.g., see FIG. 1A or 1B. Second end portions 34 and 44 also have straight angled ends having edges 33 and 43, respectively. Second end portions 34 and 44, and edges 33 and 43 preferably are contiguous with edge 23 of loop 24. First end portions 32 and 42 are cantilevered to extend distally beyond edge 21 (FIGS. 2, 3) and substantially along longitudinal axis-X. Edges 35 and 45 are connected with edge 21 of tubular wall 26 and opposed by edges 37 and 47. Arm 30 has an outer face 36 and an inner face 38. Arm 40 similarly has an outer face 46 and an inner face 48.

Similar to the previous embodiment, arms 30 and 40 preferably are elongated and plate-like extending substantially parallel with longitudinal axis-X and perpendicular to axis-Y. Arms 30 and 40 each have cantilevered distal end portions or tips 32 and 42, and proximal second end portions 34 and 44, connected to spring section 20. First end portions 32 and 42 preferably have straight angled ends having first edges 31 and 41, respectively, which are generally parallel with axis-Y. Second end portions 34 and 44 also have straight angled ends that include edges 33 and 43, respectively. Second end portions 34 and 44, and edges 33 and 43 are contiguous with edge 23 of loop 24. First end portions 32 and 42 are cantilevered to extend distally beyond edge 21 and substantially along longitudinal axis-X. Edges 35 and 45 are connected with edge 21 of tubular wall 26 and opposed by edges 37 and 47. Arm 30 has an outer face 36 and an inner face 38. Arm 40 similarly has an outer face 46 and an inner face 48.

Alternatively, as seen in FIG. 1A, ligating clip 10′ includes spring section 20 and jaw 60 as in the embodiment hereinabove disclosed. A pair of elongated beams, or arms, 30′ and 40′ are included that communicate with and extend distally from jaw 60. Each arm 30′, 40′ includes respective inner faces 38′, 48′ that are each convex and engaged along axis-X where convex inner face 38′ is adapted to complementarily engage convex inner face 48′ for occluding a vessel. When ligating clip 10′ is biased by spring section 20 in a normal first position, inner faces 38′ and 48′ are in contact with each other at least along longitudinal axis-X for occluding a vessel. Arms 30′ and 40′ further include outer faces 36′ and 46′ that are generally concave and complementary to inner faces 38′ and 48′.

Alternatively, as seen in FIG. 1B, ligating clip 10″ includes spring section 20 and jaw 60 as in the embodiment hereinabove disclosed. A pair of elongated beams, or arms, 30″ and 40″ are included that communicate and extend distally from jaw 60. Each arm 30″, 40″ includes respective inner faces 38″, 48″ that are convex and engaged along axis-X, where the convex inner faces 38″ and 48″ are juxtaposed to face one another. When ligating clip 10″ is biased by spring section 20 in a normal first position, inner faces 38″ and 48″ are in contact with each other along longitudinal axis-X for occluding a vessel. Arms 30″ and 40″ further include outer faces 36″ and 46″ that are generally concave and complementary to inner faces 38″ and 48″.

Ligating clip 10 has a first position wherein inner faces 38 and 48 are generally parallel and in substantial direct contact as a result of the bias of spring section 20. The amount of bias applied by spring section 20 can be varied depending upon the needs of the application by varying factors such as the material of loop 24, for example. In the second position, arms 30 and 40 of jaw 60 are forced open against the bias of spring section 20. Arms 30 and 40 are separated and flexibly pivot about arcuate portion 27. Arms 30 and 40 can remain generally parallel to longitudinal axis-X in the second position in this preferred embodiment, but the relationship of arms 30 and 40 can also vary in the second position such that, for example, first end portions 32 and 42 may be distanced less than or greater than second end portions 34 and 44 are distanced.

Ligating clips 10 can also include mechanical devices or features to assist the application of ligating clips 10 during minimally invasive surgery such as a pair of sockets or holes 80 on the outer edges 33 and 43, for example, in parallel with longitudinal axis-X. Sockets 80 can provide the ability for remotely spreading arms 30 and 40 in opposing directions of axis-Z and are configured to extend a suitable distance to ensure proper flexing and application of arms 30 and 40. Alternately, by way of example, instead of or with sockets 80, one could employ slots formed in edges 37 and 47 for the manipulation of arms 30 and 40, respectively or in combination with slots or holes defined in the distal edges of arms 30, 40 parallel with longitudinal axis-X.

Ligating clip 10 can be fabricated of a suitable medical grade metal, composite, or plastic material such that spring section 20 provides a flexing type movement and a bias to arms 30 and 40. The bias also provides sufficient rigidity to jaw 60 for securely clipping or occluding a vessel 70 in the first position (see FIG. 3). Spring section 20 can also be fabricated as layers of materials. For example, spring section 20 can have a metal base and a plastic coating on inner faces 38 and 48 for grip enhancement or minimizing trauma to the tissue portion being clipped. In one preferred embodiment, jaw 60 and spring section 20 are monolithically formed as one continuous element.

Referring to FIGS. 2 and 3, ligating clip 10 is shown being employed on vessel 70.

Ligating clip 10 is typically applied by positioning the longitudinal axis-X of arms 30 and 40 perpendicular to the longitudinal axis of vessel 70, which is generally parallel to axis-Y. Arms 30 and 40 are preferably configured to remain aligned with the longitudinal axis-X and with axis Y during application, but can vary in their angular relationship relative to each other and longitudinal axis-X. Preferably, arms 30 and 40 are configured to remain generally linear and not excessively bend or distort their alignment during operational use.

In addition, depending upon the strength of the bias in jaw 60 and the thickness of vessel 70 upon which ligating clip 10 is employed, arms 30 and 40 may be separated to accommodate the thickness of the compressed tissue portion of vessel 70 after application. It is recognized, however, depending upon the material(s) of construction of jaws 60, strength of the bias in spring section 20, location of vessel 70 relative to spring section 20, and the thickness of vessel 70 upon which jaw 60 is employed, a portion or portions of arms, 30 and 40 may be at least partially in direct contact with each other after being applied on vessel 70.

Ligating clip 10 is shown in operation in FIG. 2 during the application of forces against or overcoming the bias of spring section 20 and during the positioning of vessel 70 between arms 30 and 40 (See FIG. 3). Loop 24 of clip 10 applies sufficient bias to jaw 60 such that arms 30, 40 collapse vessel 70 and terminate flow therethrough. The amount of bias and area of application of arms 30 and 40 can make the application of ligating clip 10 temporary or permanent.

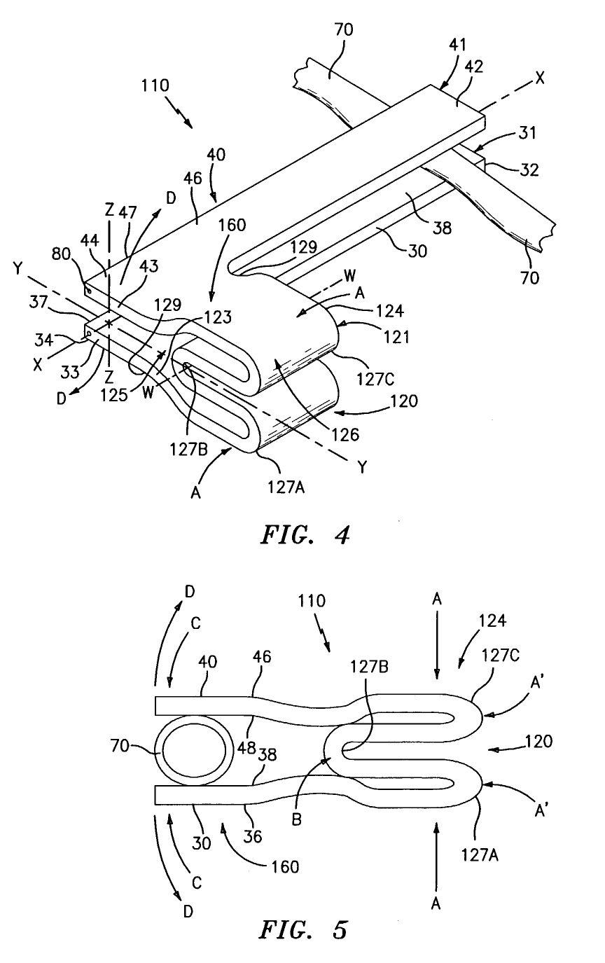

Referring now to FIG. 4, ligating clip 110, in another preferred embodiment, has a jaw 160 and a complex shaped spring section 120 including an overall loop 124 having multiple contiguous loops or bends. Jaw 160 and loop 124 define orthogonal axes X, Y, and Z. Loop 124 defines a hole 125 aligned with an axis-W and parallel to axis-X. Loop 124 includes a complex shaped spring having multiple contiguous U-shaped bends having a first edge 121 opposing a second edge 123. Loop 124 has a pair of exterior first bend portions 127A and 127C positioned on either side of or below and above a centrally or interiorly positioned second bend portion 127B. Portions 127A and 127C have convex bends facing away from axis-W such that their walls leading to the convex bends run in a direction generally parallel with axis-Y. Their walls also are generally parallel to axis-W. Portion 127B has a bend in the vicinity of axis-W that is aligned or parallel with axis-Y and has a concave face oriented in the direction of the convex faces of portions 127A and 127C. Loop 124 has outer tubular wall generally designated 126 having two ends connected respectively to arm 30 and arm 40 of jaw 160.

When viewed in a cross-section taken along axis Y and perpendicular to the longitudinal axis-X (see FIG. 5), bends 127A, 127B, and 127C define a generally wishbone shape having the function of providing spring section 120 with approximately three times the bias to jaw 160 than the bias applied by spring section 20 to jaw 60.

Similar to the previous embodiment, arms 30 and 40 preferably are elongated and plate-like extending substantially parallel with longitudinal axis-X and perpendicular to axis-Y. Arms 30 and 40 each have cantilevered distal end portions or tips 32 and 42, and proximal second end portions 34 and 44, connected to spring section 120. First end portions 32 and 42 preferably have straight angled ends having first edges 31 and 41, respectively, which are generally parallel with axis-Y. Second end portions 34 and 44 also have straight angled ends that include edges 33 and 43, respectively. Second end portions 34 and 44, and edges 33 and 43 are contiguous with edge 23 of loop 124. First end portions 32 and 42 are cantilevered to extend distally beyond edge 121 and substantially along longitudinal axis-X. Edges 35 and 45 are connected with edge 121 of tubular wall 126 and opposed by edges 37 and 47. Arm 30 has an outer face 36 and an inner face 38. Arm 40 similarly has an outer face 46 and an inner face 48.

Ligating clip 110 can also include mechanical devices or features to assist the application of ligating clip 110 during minimally invasive surgery such as a pair of sockets or holes 80 near the outer edges 33 and 43, for example, in parallel with longitudinal axis-X. Sockets 80 can provide the ability for remotely spreading arms 30 and 40 in opposing directions of axis-Z and are configured to extend a suitable distance to ensure proper flexing and application of arms 30 and 40. Alternately, by way of example, instead of or with sockets 80, one could employ slots formed in edges 37 and 47 for the manipulation of arms 30 and 40, respectively or in combination with slots or holes defined in distal edges of arms 30 and 40 parallel with longitudinal axis-X.

Ligating clip 110 in the second position, as shown in FIG. 5, has arms 30 and 40 flexing about portion 127B in opposing directions as shown by arrows-D generally along axis-Z as a result of the application of forces-A. The spreading of arms 30 and 40 against the bias provided by spring section 120 moves arms 30 and 40 into an open or spread position, thereby enabling the positioning of a tissue portion, such as vessel 70 between opened arms 30 and 40 of jaw 160. Loop 124 is configured to support the opening of jaws 160 by a manual or mechanically assisted application of forces-A in the direction of the arrows, i.e., on bends 127A and 127C. The application of forces-A yields bending at points A′ and flexing at point B which moves arms 30 and 40 in the directions of arrows-D, i.e., from the first position to a second position. The movement of arms 30 and 40 from the first position to the second position is against the bias of loop 124 as shown by arrows-C. U-shaped bends 127A and 127C have sufficient rigidity to retain their general U-shaped configuration and flex with respect to bend 127B causing arms 30 and 40 to pivot open in the direction of arrows-D. The second position is defined by arms 30 and 40 no longer being in direct contact and being displaced sufficiently for the positioning of a vessel 70 therein. Releasing forces-A causes jaw 160 to return to the first position as a result of the bias of loop 124 on arms 30 and 40 in the direction of arrows-C.

In operation, ligating clip 110 is initially in the first position and preferably biased by spring section 120 to the first position wherein arms 30 and 40 are in direct contact and parallel to longitudinal axis-X. Spring section 120 is configured for arcuate portion 127B to act as a single area of flexure urging jaws 160 to the first position to occlude vessel 70. Arms 30 and 40 are preferably biased to the first, or closed, position by spring section 120. Spring section 120 can include multiple areas of flexures, as in arcuate portions 127A, 17B, and 127C of spring section 120 configured, for providing additional bias urging jaws 160 to the first position.

Ligating clip 110 can be fabricated of a suitable medical grade metal, composite, or plastic material such that spring section 120 provides a flexing type movement and a bias to arms 30 and 40 of jaw 160 as well as sufficient rigidity in bends 127A, 127B, and 127C and jaw 160 to securely occlude or constrict vessel 70 in the first position. Spring section 120 can be fabricated as layers of materials. For example, spring section 120 can have a metal base and a plastic coating on inner faces 38 and 48 for grip enhancement or minimizing trauma to the tissue portion being clipped. In one preferred embodiment, jaw 160 and spring section 120 are fabricated as one continuous element.

Still referring to FIG. 5, ligating clip 110 is shown being employed on vessel 70. Ligating clip 110 is typically applied by positioning the longitudinal axis-X of arms 30 and 40 perpendicular to the longitudinal axis of vessel 70, which is generally parallel to axis-Y. Arms 30 and 40 are preferably configured to remain aligned with the longitudinal axis-X and with axis-Y (see FIG. 4) during application, but can vary in their angular relationship relative to each other and longitudinal axis-X. Preferably, arms 30 and 40 are configured to remain generally linear and not excessively bend or distort their alignment during operational use.

In addition, depending upon the strength of the bias in jaw 160 and the thickness of vessel 70 upon which ligating clip 110 is employed, arms 30 and 40 may be separated to accommodate the thickness of the compressed tissue portion or in direct contact with vessel 70 after application. It is recognized, however, depending upon the material(s) of construction of jaws 160, strength of the bias in spring section 120, location of vessel 70 relative to spring section 120, and the thickness of vessel 70 upon which jaw 160 is employed, a portion or portions of arms 30 and 40 may be at least partially in direct contact with each other after being applied on vessel 70.

Ligating clip 110 is shown in operation during the application of forces in the direction of arrows-A and the positioning of vessel 70 between arms 30 and 40 (See FIG. 5). Loop 124 of clip 110 applies sufficient bias in the direction of arrows-C onto jaw 160 such that arms 30, 40 collapse and terminate the flow in vessel 70 positioned therein. The amount of bias and area of application of arms 30 and 40 can make the application of ligating clip 110 temporary or permanent.

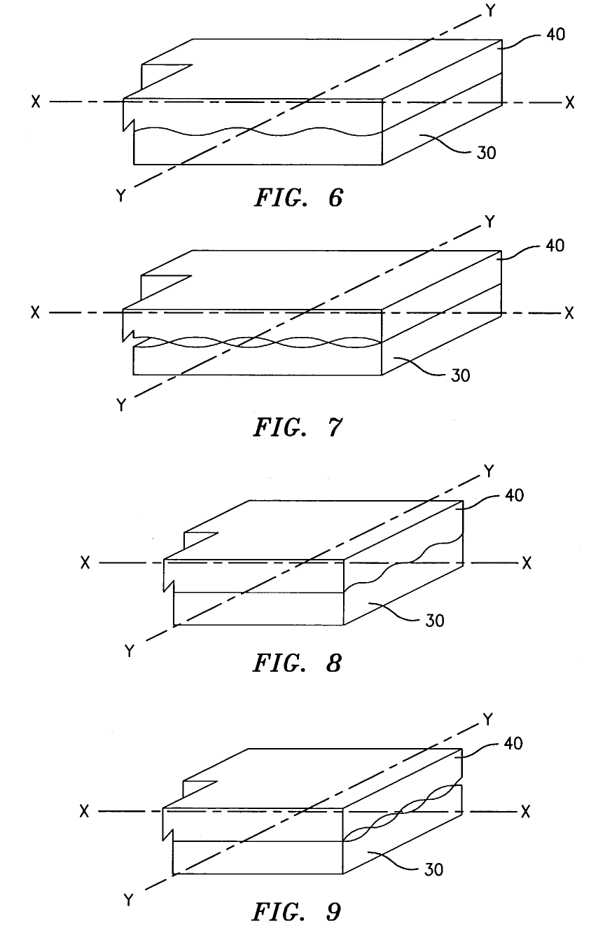

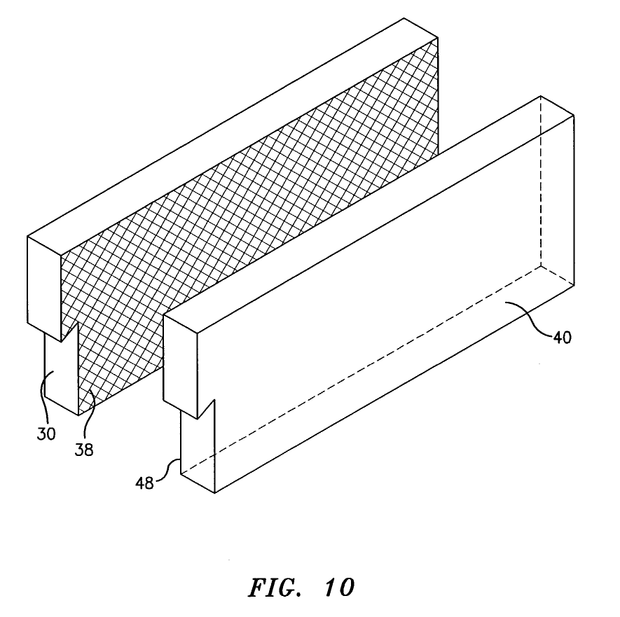

Referring now to FIGS. 6-10, inner faces 38 and 48 of clips 10, 110 can include corresponding undulations or arcuate portions that can enhance the application or gripping of inner faces 38 and 48 on a vessel (not shown). The undulations can take the form of a repeating pattern, such as a sinusoidal pattern having amplitudes having any directional orientation and configured for providing a variety of interfaces between arms 30 and 40. As shown, the interfacing of inner faces 38 and 48 may have a sinusoidal pattern aligned with axes X (FIG. 6) or Y (FIG. 8) that matingly interface to increase the surface area of contact. Alternatively, the repeating pattern may be offset (FIGS. 7 and 9) such that there is a peak-to-peak interface or a variation thereof such as a slope-to-slope interface. Inner faces 38 and 48 can include other geometric patterns such as knurling (FIG. 10) to enhance the gripping of a figure portion. The dimensions of arms 30 and 40 in the direction of axis-Y and axis-X can be varied to enhance the gripping or interfacing area so as to spread the applied clipping load or in the alternative to concentrate the applied forces over an axial length of a vessel. Similarly, tips 32 and 42 can have any geometric shape.

Although the illustrative embodiments of the present disclosure have been described herein with reference to the accompanying drawings, it is to be understood that the disclosure is not limited to those precise embodiments, and that various other changes and modifications may be affected therein by one skilled in the art without departing from the scope or spirit of the disclosure. All such changes and modifications are intended to be included within the scope of the disclosure.

What is claimed is:

1. A surgical clip for occluding a vessel, the surgical clip comprising:
a jaw comprised of first and second arms that define a longitudinal axis, each arm having a proximal and distal end, and an inner face and an outer face; and
a spring section in communication with the proximal ends of the jaw section, the spring section being for biasing the arms into a normal first position wherein the inner face of the first arm abuts at least a portion of the inner face of the second arm, the spring section including at least one arcuate wall that defines at least arcuate loop.

2. The surgical clip of claim 1, wherein the inner face of the first arm abuts the inner face of the second arm substantially along the longitudinal axis.

3. The surgical clip of claim 1, wherein each arm includes a socket disposed at the distal end of each arm.

4. The surgical clip of claim 1, wherein the inner faces of the first and second arms are knurled.

5. The surgical clip of claim 1, wherein the inner faces of the first and second arms have a sinusoidal pattern.

6. The surgical clip of claim 5, wherein the sinusoidal pattern of the first arm is complementary to the sinusoidal pattern of the second arm.

7. The surgical clip of claim 1, wherein the spring section is formed from a unitary continuous wall that forms the at least one arcuate loop, the at least one arcuate loop being a bulbous loop having a closed loop end portion facing away from the jaw, and an opposed end formed of converging first and second wall portions, one in communication with the proximal end of the first arm and the other in communication with the proximal end of the second arm.

8. The surgical clip of claim 1, wherein the inner face of the first arm includes a substantially convex portion directed toward the second arm and the inner face of the second arm includes a juxtaposed substantially convex portion directed toward the first arm, each of the convex portions being engaged with one another along the longitudinal axis of the arms.

9. The surgical clip of claim 1, wherein the inner face of the first arm includes a substantially convex portion and the inner face of the second arm includes a complimentary substantially convex portion, each of the convex portions are engaged with one another along the longitudinal axis of the arms.

10. A surgical clip for occluding a vessel, the surgical clip comprising:
a jaw comprised of first and second elongated cantilevered beams that define a longitudinal axis, each beam having a proximal end and a distal end; and
a spring section in communication with the proximal end of the beams for biasing the beams into a normal first position wherein the inner face of the first beam abuts at least a portion of the inner face of the second beam, the spring section including first and second exterior arcuate members, the first exterior arcuate member being in communication with the proximal portion of the first beam and the second exterior arcuate member being in communication with the proximal portion of the second beam, the first and second exterior arcuate members forming a third arcuate member therebetween.

11. The surgical clip of claim 10, wherein the first and second beams have an inner face and an outer face, the inner face of the first beam abuts the inner face of the second beam substantially along the longitudinal axis.

12. The surgical clip of claim 10, wherein the first and second exterior arcuate members include respective first and second loops, the first and second loops each having an opening facing the proximal ends of the first and second beams.

13. The surgical clip of claim 12, wherein the first and second exterior loops form a third arcuate member in the form of a third loop, the third loop being located interior of the first and second loops and having an opening that faces substantially 180° away from the first and second openings.

14. The surgical clip of claim 13, wherein the first, second, and third loops are spaced apart from one another and are transversely aligned.

15. The surgical clip of claim 14, wherein the first, second, and third loops are flattened and have substantially parallel sidewalls.

16. The surgical clip of claim 15, wherein the first, second, and third loops, and the jaw are a continuous band of material.

17. A surgical clip for occluding a vessel, the surgical clip comprising:
a jaw comprised of first and second arms that define a longitudinal axis, each arm having a proximal end and a distal end, and an inner face and an outer face; and
a spring section in communication with the proximal ends of the arms for biasing the arms into a normal first position wherein the inner face of the first arm abuts at least a portion of the inner face of the second arm, the spring section being U-shaped and formed of a unitary continuous wall that forms multiple contiguous bends, including first and second exterior U-shaped loops each having a convex exterior surface facing away from the jaw, and a third interior loop disposed between and extending from the first and second loops, the-third interior loop having an exterior concave surface facing the jaws, and the exterior walls of the first and second exterior loops extending into the first and second proximal end portions of the arms of the jaw.

18. The surgical clip of claim 17, wherein the first, second, and third loops are formed by substantially U-shaped walls and are spaced apart from each other.

19. The surgical clip of claim 17, wherein portions of the substantially U-shaped walls are substantially parallel to each other.

20. The surgical clip of claim 18, wherein the substantially U-shaped walls of the first, second, and third loops are transversely aligned.

21. The surgical clip of claim 18, wherein the substantially parallel portions of the first and second exterior loops communicate with a transitional wall portion, the transitional wall portion having an inward bend that extends toward and engages the third interior loop.

22. The surgical clip of claim 21, wherein first and second loops are configured such that when the proximal end portions of the arms are opened against their bias, the first and second loops move toward each other.
